(19) United States
(12) Patent Application Publication
(10) Pub. No.: US 2026/0248500 A1
(43) Pub. Date: Aug. 27, 2026

(54) LOCKING DEVICE, SYSTEM AND METHOD OF USE THEREOF

(71) Applicant: MVRx, Inc., San Mateo, CA (US)

(72) Inventors: David R. Tholfsen, San Leandro, CA (US); Patrick P. Wu, San Carlos, CA (US)

(21) Appl. No.: 18/993,959

(22) PCT Filed: Aug. 8, 2023
(86) PCT No.: PCT/US2023/029706

§ 371 Date: Jan. 13, 2025

Related U.S. Application Data
(60) Provisional application No. 63/396,551, filed on Aug. 9, 2022.

(30) Foreign Application Priority Data
Aug. 9, 2022 (CN) 202210948160.3
Aug. 18, 2022 (CN) 202210990971.X

(51) Int. Cl. A61B 17/04; A61B 17/00

(52) U.S. Cl. CPC A61B 17/0487; A61B 2017/00867

(57) ABSTRACT

Systems, methods and associated devices for locking a tensioning element, such as a bridging element, suture, wire or string, during a medical procedure, such as placement of a medical implant in a subject are provided herein. The systems, methods and devices described herein are particularly useful for delivery and deployment of heart implants for reshaping a heart valve annulus for treatment of a heart disorder, such as mitral valve regurgitation.

### BACKGROUND OF THE INVENTION

### CROSS-REFERENCE TO RELATED APPLICATIONS

This application claims the benefit of priority under 35 U.S.C. 119(e) of U.S. Provisional Application No. 63/396,551 filed Aug. 9, 2022. The disclosure of the prior application is considered part of and is herein incorporated by reference in the disclosure of this application in its entirety.

### FIELD OF THE INVENTION

The present invention relates generally to medical devices and procedures, and more particularly to a device, system and method for locking a suture or wire to prevent movement of the suture or wire during a medical procedure.

### BACKGROUND INFORMATION

Use of suture locks to secure a suture and maintain a holding force are used during conventional medical procedures. In some instances, the lock is a three part system where there is a jaw, an outer shell and a compression spring that keeps the edges of the jaw and shell close together. To open the lock the spring must be compressed to push the edges of the jaw and shell apart. This design is limited by how much the edges of the jaw and shell can be pulled apart from each other. This in turn limits the size of the suture/tensioning element or other components that are bonded to the suture.

As such, there is a need for improved lock designs which are easier to manipulate and are useful in a wider range of applications.

### SUMMARY OF THE INVENTION

Embodiments of the invention are directed to systems, methods and associated devices for locking a suture or wire during a medical procedure, such as placement of a medical implant in a subject. The systems, methods and devices described herein are particularly useful for delivery and deployment of heart implants for reshaping a heart valve annulus for treatment of a heart disorder, such as mitral valve regurgitation.

In some embodiments, the invention provides a locking device that includes:
a body having a first opposing member connected to a second opposing member by a hinge element, each opposing member being resiliently movable about the hinge element between a locked position and an unlocked position, wherein the hinge is configured to maintain a compressive force between opposing surfaces of the opposing members;
a channel disposed adjacent the body and extending partially or entirely through a thickness of the body; and
a slot disposed adjacent the channel, the slot being open when the device is transitioned from the locked position to the unlocked position.

In embodiments, the opposing surfaces are separated from one another in the unlocked position and the slot is open and are in close proximity to one another in the locked position to apply a compressive force to an object within the slot and the slot is closed.

In some embodiments, the invention provides a locking device that includes:
a disc body having an outer perimeter surface and a first opposing member connected to a second opposing member by a hinge element, each opposing member being resiliently movable about the hinge element between a locked position and an unlocked position, wherein the hinge formed by a strip of the body connecting the first and second opposing members and configured to maintain a compressive force between opposing surfaces of the opposing members;
a channel disposed adjacent the body and extending through a thickness of the body; and
a slot disposed adjacent the channel, the slot being open when the device is transitioned from the locked position to the unlocked position.

In embodiments, the slot is defined by a groove in the opposing surface of the first opposing member or the opposing surface of the second opposing member and the slot is shaped to accommodate a ridge formed on the opposing surface, and the hinge, slot and channel are aligned along a midline of the body in the locked position with the channel being disposed in the center of the body. In further embodiments, the opposing surfaces are separated from one another in the unlocked position and the slot is open, and the opposing surfaces are in close proximity to one another and apply a compressive force to an object within the slot in the locked position and the slot is closed.

In some embodiments, the invention provides a locking assembly. In embodiments, the locking assembly includes 2 or more locking devices of the invention, wherein each locking device is arranged such that the hinge and channel of each locking device are aligned.

In some embodiments, the invention provides a locking system. In embodiments, the locking system includes:
the locking device or the locking assembly of the invention; and
a medical device detachably coupled to the locking device or locking assembly.

In some embodiments, the invention provides a method of performing a surgical procedure. In embodiments, the method includes:
advancing the locking device or locking assembly to a location within a subject;
transitioning the locking device or locking assembly to the unlocked position;
tensioning a tensioning element traversing the slot; and
transitioning the locking device or locking assembly to the locked position.

In some embodiments, the invention provides a method of treating mitral valve regurgitation in a subject by reshaping an atrial heart chamber of the subject. In embodiments, the method includes:
advancing the locking device or locking assembly of the disclosure to a first location of the heart within the subject, wherein the locking device or locking assembly is coupled to an expandable anchor implant;
deploying the expandable anchor implant at the first location by tensioning the tensioning element when the locking device or locking assembly is in the unlocked position;
transitioning the locking device or locking assembly to the locked position;
detaching the catheter from the locking device or locking assembly;
deploying a second expandable anchor at a second location of the heart, wherein the first and second expandable anchors are coupled via a bridging element traversing the heart chamber; and
tensioning the bridging element to reshape the heart chamber, thereby treating mitral valve regurgitation in the subject.

### BRIEF DESCRIPTION OF THE FIGURES

FIG. 1 is a perspective view of a locking device in the locked position in an embodiment of the invention.

FIG. 2 is a perspective view of the locking device depicted in FIG. 1 in the unlocked position via application of an expanding member.

FIG. 3 illustrates a locking assembly in the unlocked position in an embodiment of the invention.

FIG. 4 depicts the locking assembly of FIG. 3 in the locked position.

FIG. 5 illustrates a locking assembly in an embodiment of the invention.

FIG. 6 illustrates a locking device in the locked position in an embodiment of the invention.

FIG. 7 depicts the locking assembly of FIG. 6 in the unlocked position.

FIG. 8 depicts the locking assembly of FIG. 6 in the unlocked position.

FIG. 9 depicts the locking assembly of FIG. 6 in the locked position.

FIG. 10 depicts the locking assembly of FIG. 6 in the locked position.

FIG. 11 illustrates a locking assembly in the locked position in an embodiment of the invention.

FIG. 12A depicts the locking assembly of FIG. 9 in the unlocked position.

FIG. 12B depicts the locking assembly of FIG. 9 in the unlocked position with a suture traversing the slot.

FIG. 12C depicts the locking assembly of FIG. 9 transitioning from the unlocked position to the locked position by removal of the expanding member.

FIG. 12D depicts the locking assembly of FIG. 9 in the locked position.

FIG. 13 illustrates a locking assembly in the locked position in an embodiment of the invention.

FIG. 14A depicts the locking assembly of FIG. 11 in the unlocked position.

FIG. 14B depicts the locking assembly of FIG. 11 in the unlocked position with a suture traversing the slot.

FIG. 14C depicts the locking assembly of FIG. 11 transitioning from the unlocked position to the locked position by removal of the expanding member.

FIG. 14D depicts the locking assembly of FIG. 11 in the locked position.

FIG. 15 illustrates a locking system in an embodiment of the invention.

FIG. 16 illustrates a locking system in an embodiment of the invention.

FIG. 17 is an expanded view of the locking system depicted in FIG. 16.

### DETAILED DESCRIPTION OF THE INVENTION

The present invention relates to systems, methods and associated devices for locking a tensioning element, such as a bridging element, suture, wire or string, during a medical procedure, such as placement of a medical implant in a subject. The systems, methods and devices described herein are particularly useful for delivery and deployment of heart implants for reshaping a heart valve annulus for treatment of a heart disorder, such as mitral valve regurgitation.

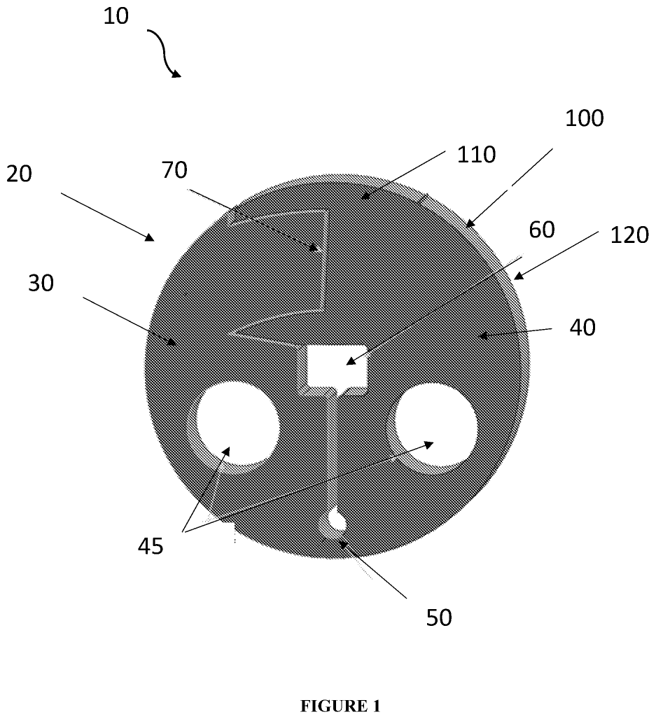

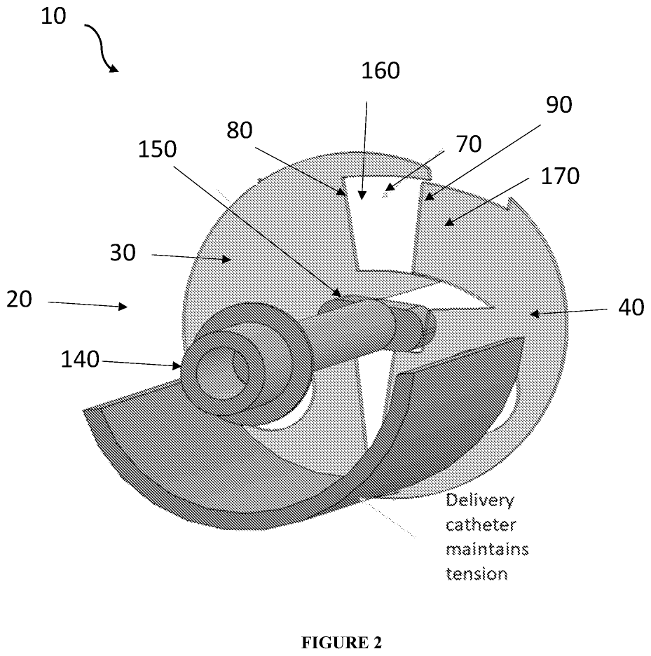

As such, embodiments of the invention are directed to a locking device for reversibly securing a tensioning element, such as a suture or wire, under tension during a surgical procedure. The device is capable of securing the tensioning element under tension without slipping of the tensioning element which, for example, would result in the incorrect deployment of a medical implant, such as a septal implant. With reference to FIGS. 1 and 2, the locking device 10 includes a body 20 having a first opposing member 30 connected to a second opposing member 40 by a hinge element 50, a channel 60 disposed adjacent the body 20 and extending partially or entirely through a thickness of the body, a slot 70 disposed adjacent the channel 60, and optionally one or more aligning holes 45. Each opposing member (30, 40) is resiliently movable about the hinge element 50 between a locked position as shown in FIG. 1, and an unlocked position as shown in FIG. 2. In embodiments, the hinge element 50 is configured to maintain a compressive force between opposing surfaces (80, 90) of the opposing members (30, 40). As shown in FIG. 2, the slot 70 is open when the device 10 is transitioned from the locked position to the unlocked position.

In embodiments, the opposing surfaces (80, 90) are separated from one another in the unlocked position and the slot 70 is open as shown in FIG. 2. In the locked position, the opposing surfaces (80, 90) are in close proximity to one another to apply a compressive force to an object within the slot 70 and the slot 70 is closed as shown in FIG. 1. As such, the opposing surfaces (80, 90) are operable to apply a compressive force to a tensioning element traversing the slot 70 in the locked position.

In embodiments, the body 20 has an outer perimeter surface 100 surrounding the first opposing member 30, the second opposing member 40, and the hinge element 50 connecting the opposing members. In embodiments, the body 20 is defined by a planar substrate having a front surface 110 and an opposing back surface 120, the surfaces being bound by the outer perimeter surface 100.

While the Figures illustrate embodiments in which the opposing front and back surfaces (110, 120) are circular, it will be understood that the surfaces may be virtually any geometric shape. For example, the surfaces may be circular, elliptical, oval, square, rectangular, triangular, hexagonal and the like.

In embodiments, the locking device 10 includes a channel 60 that traverses the entire thickness of the body 20. As shown in FIG. 1, when the device is in the locked position, the channel 60 has a rectangular or square shape. The channel 60 is configured to engage and interact with an expanding member 140 having a distal tip 150 sized to fit within the channel 60 in when the device is in the locked position and transition the device to the unlocked position by rotating the distal tip 150 of the expanding member 140 within the channel 60 as shown in FIG. 2.

It will be appreciated that the channel 60 and distal tip 150 of the expanding member may have a variety of corresponding shapes while maintaining the same functionality of interaction to transition the device from the locked position to the unlocked position. For example, in embodiments, the channel is rectangular, square, triangular, hexagonal, ovoid or ellipsoid and the distal tip is sized and shaped to be inserted into the channel when the device is in the locked position and expand the channel and slot to transition the device to the unlocked position.

During operation of the locking device 10, transitioning of the device to the unlocked position opens the slot 70 such that the tensioning member may freely move through the open slot of the device. FIG. 2 shows the device in the unlocked position in which the slot 70 is open. In embodiments, the slot 70 is defined by a groove formed in one of the opposing surfaces of an opposing member which receives a ridge formed on the opposing surface of the other opposing member. FIG. 2 shows the slot 70 being defined by a groove 160 formed in the opposing surface 80 of the first opposing member 30 which receives a ridge 170 formed on the opposing surface 90 of the second opposing member 40.

In various embodiments, the hinge element 50, channel 60 and slot 70 are symmetrically aligned along the midline of the device 10 with the channel being centrally located and the hinge and slot being on opposing sides of the channel when the device is in the locked position as shown in FIG. 1. This allows multiple locking devices 10 to be linked together to form a locking assembly.

As such, in embodiments, the invention provides a locking assembly. In embodiments, the locking assembly includes 2, 3, 4, 5, 6, 7, 8, 9, 10 or more locking devices aligned in succession. In some embodiments, each locking device is arranged such that the hinge and channel of each locking device are aligned. In embodiments, each locking device is arranged front to back in an alternating manner. In embodiments, each locking device is arranged back to back in an alternating manner. In embodiments, each locking device is arranged front to front in an alternating manner. In embodiments, each locking device is arranged back to back, front to front, back to back and so on.

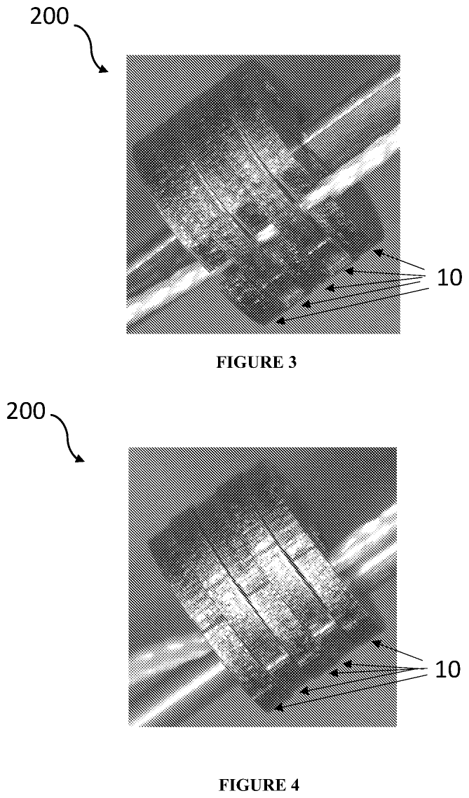

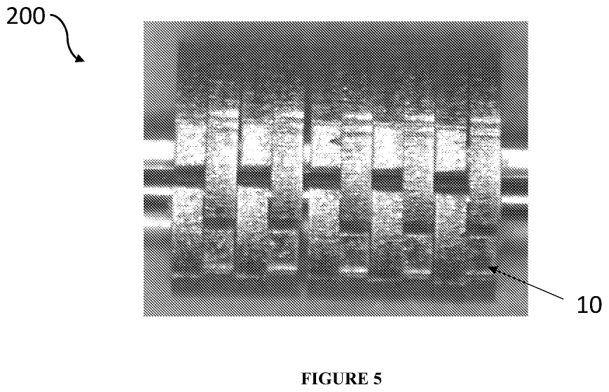

FIGS. 3-5 show locking assemblies 200 that include multiple locking devices 10. The individual locking devices 10 that comprise a locking assembly 200 may be optionally coupled to one another in an alternating fashion such that the slot, channel and hinge element of each device are aligned along the longitudinal axis of the assembly and the back surface of one device faces the back surface of another device as shown in FIGS. 3-5. This allows the individual slots of each device to be offset from one another when the assembly is in the locked position (e.g., each individual locking device is in the locked position) resulting in increased gripping of the tensioning element that traverses the slots to prevent slipping of the tensioning element when the element is under tension during a surgical procedure.

In embodiments, the compressive force applied by the opposing surfaces on the tensioning element when the device 10 is in the locked position is generated by the hinge element 50. For example, in some embodiments, at least the hinge element 50 is composed of a resilient shape memory material having a memory that naturally urges that opposing surfaces toward each other to grip and object. In embodiments, the shape memory material of the hinge element is configured to resiliently return to the locked position from the unlocked position. Such materials include polymers, composites, ceramics and alloys, such as nickel-titanium (NiTi) alloy, i.e., nitinol, and steel.

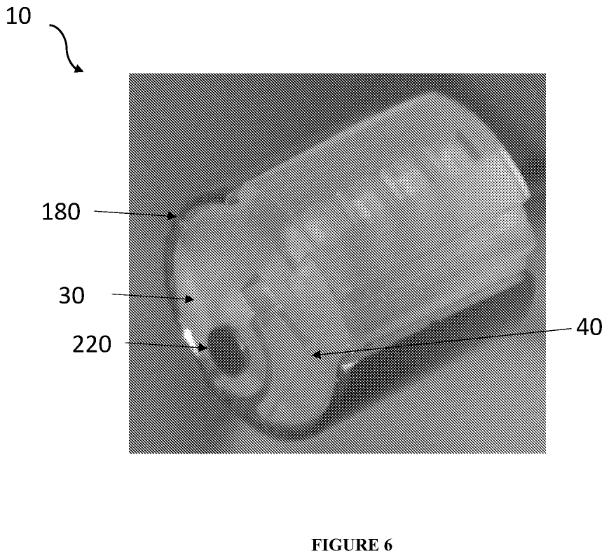

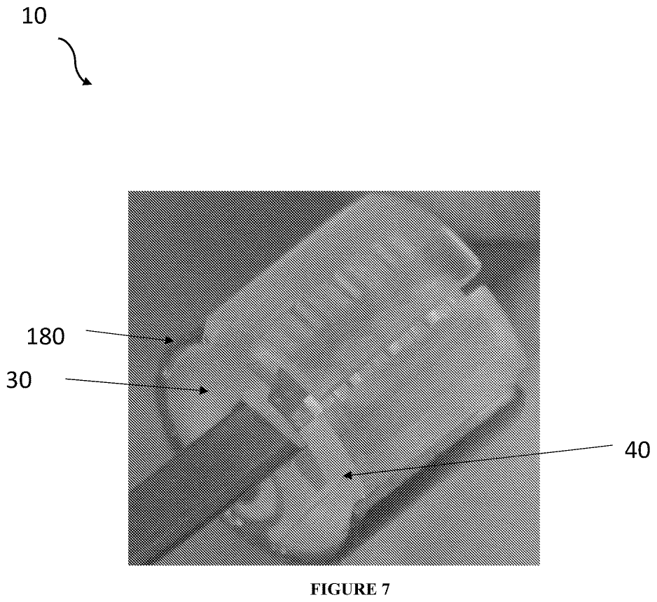

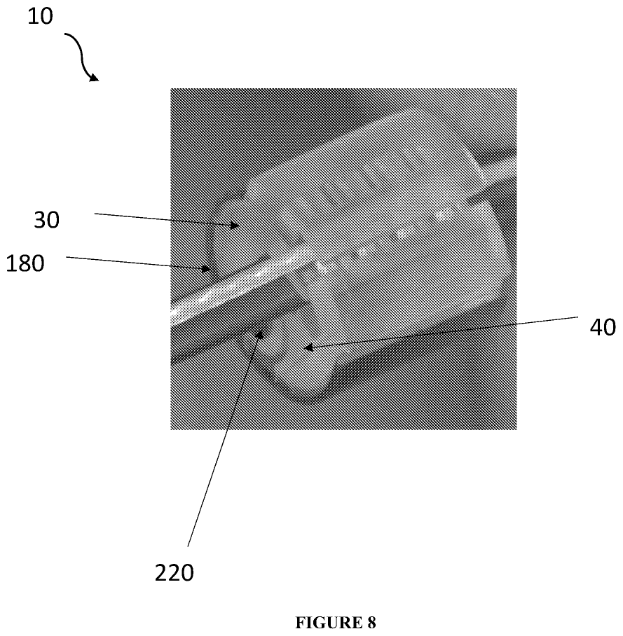

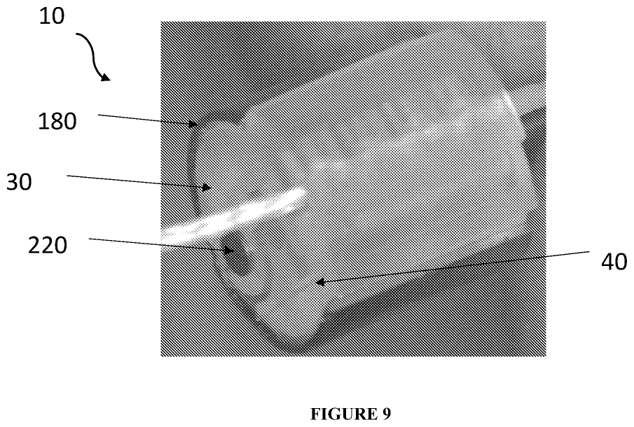

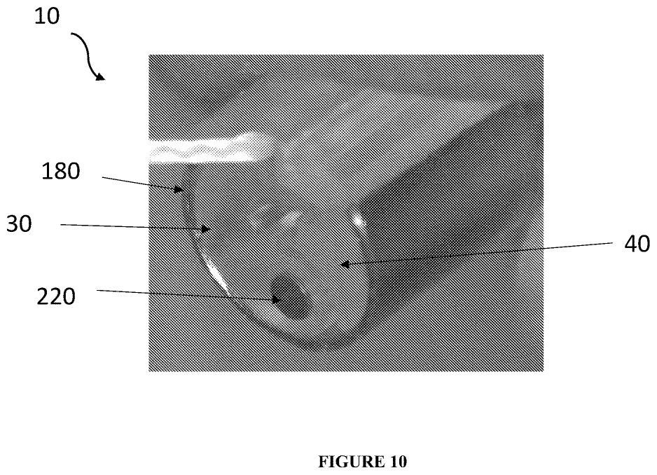

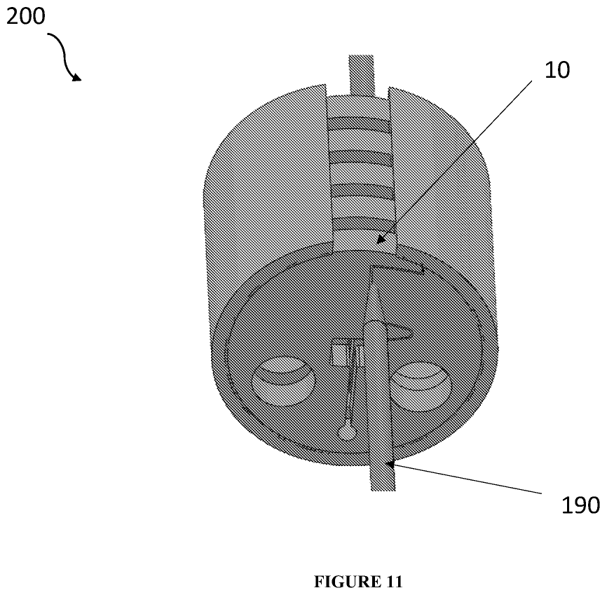

In embodiments, the hinge element 50 need not be formed of a shape memory material. In such embodiments, the compressive force applied by the opposing surfaces (80, 90) on the tensioning element when the device 10 is in the locked position is generated by a collar 180 that at least partially surrounds the outer perimeter surface 100 as shown in FIGS. 6-11. For example, in some embodiments, the collar 180 is composed of a resilient shape memory material having a memory that naturally urges that opposing surfaces toward each other to grip and object. In embodiments, the shape memory material of the hinge element is configured to resiliently return to the locked position from the unlocked position. Such materials include polymers, composites, ceramics and alloys, such as nickel-titanium (NiTi) alloy, i.e., nitinol, and steel.

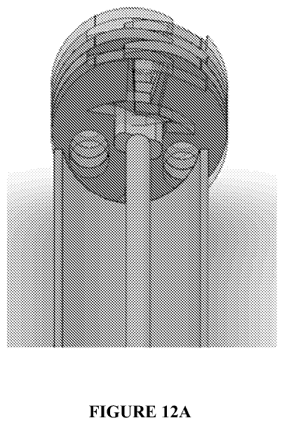

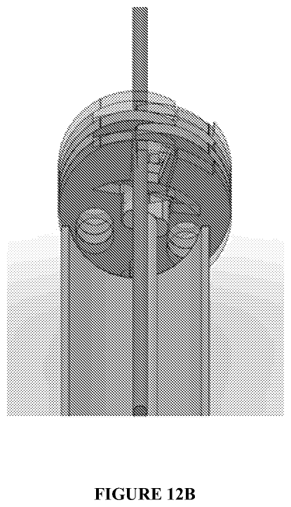

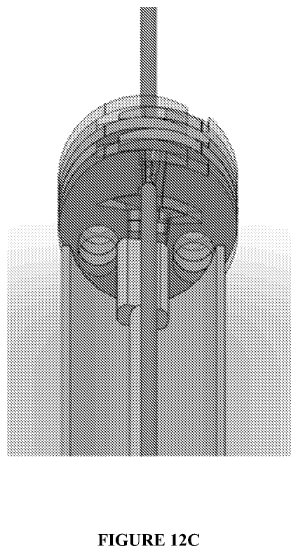

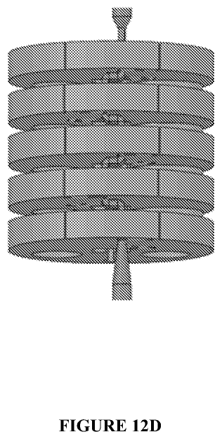

In embodiments, the hinge element 50 and the collar 180 are both formed of shape memory material. FIG. 11 shows a locking assembly 200 that includes a plurality of individual locking devices 10 as depicted in FIGS. 1 and 2, aligned in an alternating manner and surrounded by the collar 180. FIG. 11 shows the assembly 200 in the locked position gripping a tensioning element 190 that traverses the respective slots of the individual locking devices 10. During operation, the expanding member 140 is inserted into one or more channels 60 to expand the slots 70 and transition the assembly 200 to the unlocked position as shown in FIG. 12A. The tensioning element 190 is inserted through the respective slots of the assembly while the assembly 200 is unlocked with the expanding member 140 being in engagement with the one or more channels 60 as shown in FIG. 12B. The expanding member 140 is then removed from the assembly 200 once the tensioning element 190 is tensioned to deploy an implant for example, and the assembly 200 automatically transitions from the unlocked position to the locked position as shown in FIG. 12C. FIG. 12D shows the assembly 200 in the locked position (without the collar) with the tensioning element 190 being gripped with the individual slots of each locking locking device 10 of the assembly.

In embodiments, the locking device does not include a hinge element. For example, the locking device includes opposing members (30, 40) that are separate and contained within the collar 180 without a hinge. The opposing members are separate from one another and the orientation and shape of the collar (e.g., c-shape) allows the device to be transitioned from the locked position to the unlocked position. The compressive force applied by the opposing surfaces (80, 90) on the tensioning element when the device is in the locked position is generated by the collar 180 that partially surrounds the body 20 with the opening of the collar being disposed adjacent the slot 20 and the closed portion of collar being disposed opposite the slot.

FIGS. 6-10 and 13 illustrate an embodiment of the device 10 that includes opposing members (30, 40) and the collar 180. Each opposing member includes multiple opposing surfaces of grooves and ridges that form the slot, each opposing member being connected by a hinge element that includes a pin 220 that traverses the length of the device. The opposing members freely rotate about the pin 220 of the hinge element to allow the device to transition between the locked and unlocked position. The compressive force applied by the opposing surfaces of the opposing members (30, 40) on the tensioning element when the device is in the locked position is generated by the collar 180.

In embodiments, the locking device 10 or locking assembly 200 is detachably coupled to medical device, such as a delivery catheter or medical implant. As such, embodiments of the invention provide a locking system that includes the locking device or locking assembly of the disclosure and a medical device detachably coupled to the locking device or locking assembly.

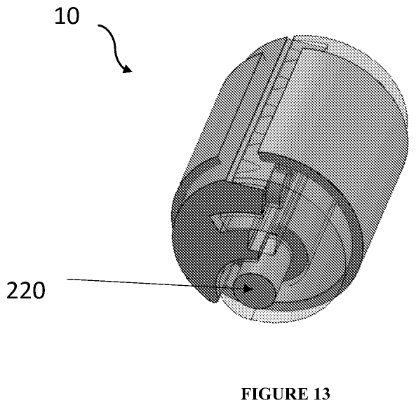

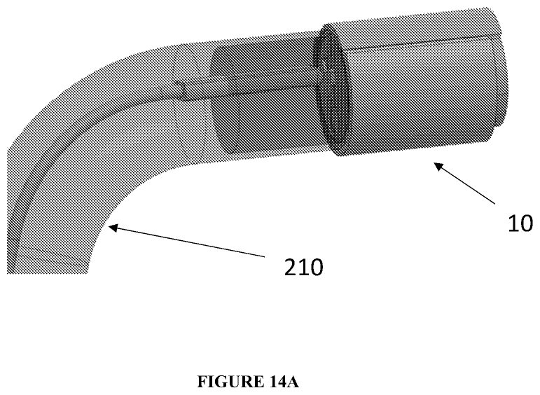

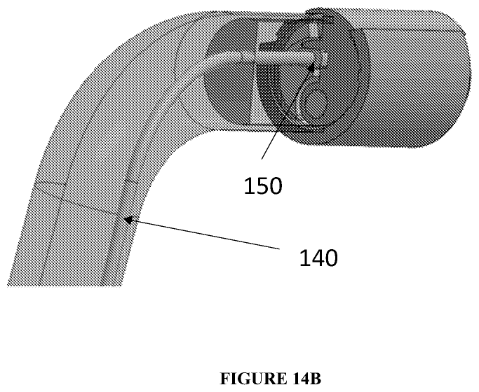

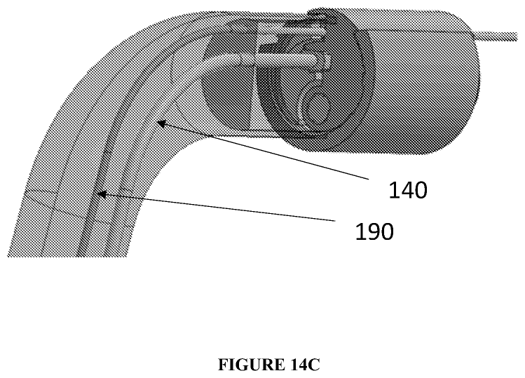

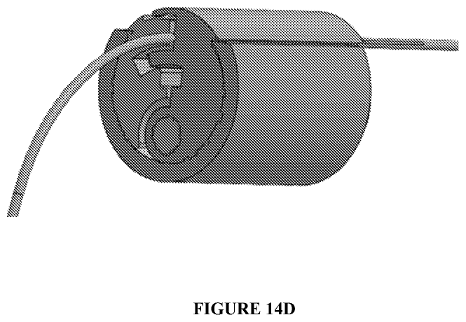

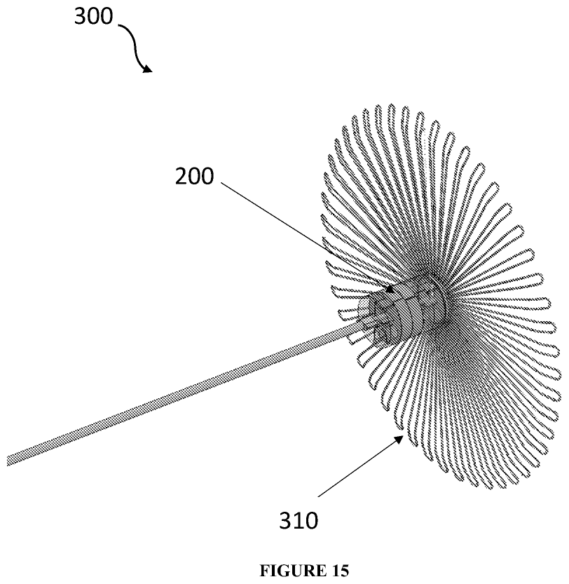

FIGS. 14A-14D illustrate delivery of the locking device 10 depicted in FIG. 13 via a delivery catheter 210. FIG. 14A shows the locking device 10 in the locked position and coupled to the delivery catheter 210. Upon delivery to a desired location, the distal tip 150 of the expanding member 140 is inserted into the channel 60 to expand the locking device and transition the device from the locked position to the unlocked position as shown in FIG. 14B. A tensioning element 190 is passed through the open slots of the locking device 10 and the delivery catheter 210 when the locking device is in the unlocked position as shown in FIG. 14C. Once the tensioning element 190 is appropriately tensioned, the expanding member is removed and the locking device 10 transitions to the closed position gripping the tensioning element 19 as shown in FIG. 14D. The delivery catheter can then be uncoupled from the locking device if desired.

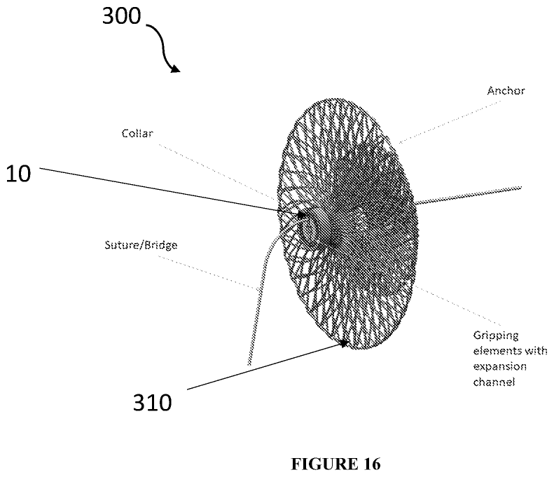

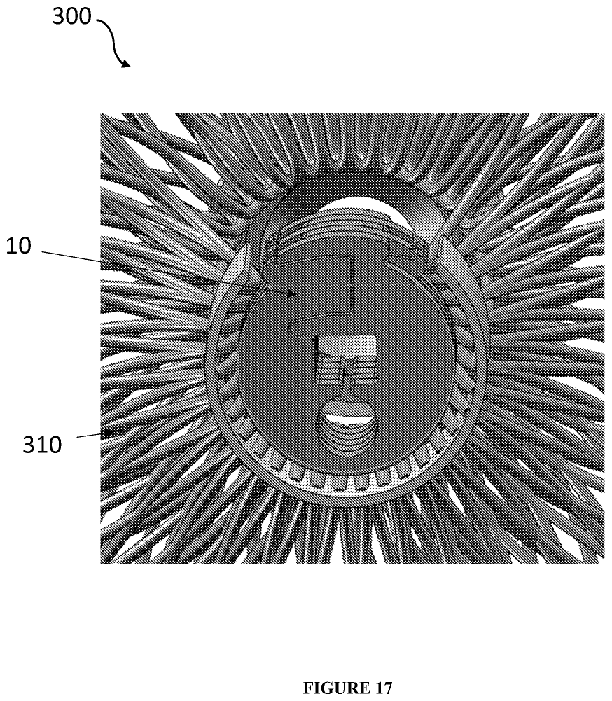

FIGS. 15-17 illustrate a locking system 300 in an embodiment of the invention in which the locking device 10 or locking assembly 200 is coupled to an expandable medical implant, e.g., an expandable septal anchor 310.

In embodiments, the locking system 300 includes a catheter and/or tissue anchor as described in U.S. patent application Ser. Nos. 17/544,768 and 17/544,696.

In embodiments, the locking device 10 or locking assembly 200 is coupled to a catheter or tissue anchor as described in U.S. patent application Ser. Nos. 17/544,768 and 17/544,696.

Embodiments of the invention are directed to methods for performing a surgical procedure using the locking device, locking assembly or locking system of the disclosure. In one embodiment, the method includes:
advancing the locking device or locking assembly to a location within a subject;
transitioning the locking device or locking assembly to the unlocked position;
tensioning a tensioning element traversing the slot; and
transitioning the locking device or locking assembly to the locked position.

In some embodiments, the surgical procedure includes deploying a medical implant, such as a tissue anchor an expandable tissue anchor. In some embodiments, the surgical procedure includes deploying a septal wall anchor or expandable septal wall anchor. In some embodiments, the surgical procedure includes deploying an anchor system to cardiac tissue. In some embodiments, the surgical procedure includes deploying a medical implant, such as a tissue anchor to treat mitral valve regurgitation as described in U.S. patent application Ser. Nos. 17/544,768 and 17/544,696.

In one embodiment, the method is for treating mitral valve regurgitation in a subject by reshaping an atrial heart chamber of the subject. The method includes:
advancing the locking device or locking assembly of the disclosure to a first location of the heart within the subject, wherein the locking device or locking assembly is optionally coupled to a delivery catheter and/or an expandable anchor implant;
deploying the expandable anchor implant at the first location by tensioning the tensioning element when the locking device or locking assembly is in the unlocked position;
transitioning the locking device or locking assembly to the locked position;
detaching the catheter from the locking device or locking assembly;
deploying a second expandable anchor at a second location of the heart, wherein the first and second expandable anchors are coupled via a bridging element traversing the heart chamber; and
tensioning the bridging element to reshape the heart chamber, thereby treating mitral valve regurgitation in the subject.

The foregoing is considered as illustrative only of the principles of the invention. The embodiments herein disclosed merely exemplify the invention which may be embodied in other specific structures. While embodiments have been described, the details may be changed without departing from the invention. Further, most of the inventions are shown in simple forms to illustrate elemental function and features and may be combined to a final embodiment that uses one or more elements combined into a single device. Furthermore, since numerous modifications and changes will readily occur to those skilled in the art, the invention is not limited to the construction and operation shown and described in the preferred embodiments except as limited by the claims.

Although the invention has been described with reference to the above disclosure, it will be understood that modifications and variations are encompassed within the spirit and scope of the invention. Accordingly, the invention is limited only by the following claims.

In the claims:

1. A locking device comprising:
a body having a first opposing member connected to a second opposing member by a hinge element, each opposing member being resiliently movable about the hinge element between a locked position and an unlocked position, wherein the hinge is configured to maintain a compressive force between opposing surfaces of the opposing members;
a channel disposed adjacent the body and extending partially or entirely through a thickness of the body; and
a slot disposed adjacent the channel, the slot being open when the device is transitioned from the locked position to the unlocked position,
wherein the opposing surfaces are separated from one another in the unlocked position and the slot is open, and
wherein the opposing surfaces are in close proximity to one another in the locked position to apply a compressive force to an object within the slot and the slot is closed.

2. The locking device of claim 1, wherein the body has an outer perimeter surface.

3. The locking device of claim 2, wherein the body is defined by a planar substrate having a front surface and an opposing back surface, the surfaces being bound by the outer perimeter surface.

4. The locking device of claim 3, wherein the opposing front and back surfaces are circular, elliptical, square, rectangular or triangular.

5. The locking device of claim 1, wherein the body is composed of a resilient shape memory material.

6. The locking device of claim 5, wherein the shape memory material is a composite, ceramic, alloy or polymer.

7. The locking device of claim 6, wherein the alloy is nitinol or steel.

8. The locking device of claim 5, wherein the shape memory material is configured to resiliently return to the locked position from the unlocked position.

9. The locking device of claim 1, wherein the channel traverses the entire thickness of the body.

10. The locking device of claim 9, wherein the channel is disposed in the center of the body.

11. The locking device of claim 1, wherein the slot is defined by a groove in the opposing surface of the first opposing member or the opposing surface of the second opposing member.

12. The locking device of claim 11, wherein the slot is shaped to accommodate a ridge formed on the opposing surface.

13. The locking device of claim 1, wherein the slot is disposed off center in the body.

14. The locking device of claim 1, wherein the hinge is formed by a strip of the body connecting the first and second opposing members, and wherein the strip is coextensive with the outer perimeter surface.

15. The locking device of claim 1, wherein the channel is square or rectangular shaped when in the locked position.

16. The locking device of claim 1, wherein the opposing surfaces are operable to apply a compressive force to a tensioning element traversing the slot in the locked position.

17. The locking device of claim 16, wherein the tensioning element is a suture, wire or string.

18. The locking device of claim 2, further comprising an outer collar member at least partially surrounding the outer perimeter surface.

19. The locking device of claim 18, wherein the outer collar member extends along the entire thickness of the body.

20. The locking device of claim 18, wherein outer collar member is composed of a resilient shape memory material.

21. The locking device of claim 20, wherein the shape memory material is a composite, ceramic, alloy or polymer.

22. The locking device of claim 21, wherein the alloy is nitinol or steel.

23. The locking device of claim 1, wherein the body is disc shaped and the slot, channel and hinge are disposed along the midline of the disc in the locked position.

24-53. (canceled)
